(19) United States
(12) Patent Application Publication
(10) Pub. No.: US 2017/0183738 A1
(43) Pub. Date: Jun. 29, 2017

(54) Process, Apparatus or System and Kit for Classification of Tumor Samples of Unknown and/or Uncertain Origin and Use of Genes of the Group of Biomarkers

(71) Applicants: Marcos Tadeu dos SANTOS, Sao Paulo (BR); Ramon Oliveira VIDAL, Itabuna (BR); Bruno Feres de SOUZA, Sao Luiz (BR); Flavio Mavignier CARCANO, Barretos (BR); Cristovam Scapulatempo NETO, Barretos (BR); Cristiano Ribeiro VIANA, Barretos (BR); Andre Lopes CARVALHO, Barretos (BR)

(72) Inventors: Marcos Tadeu dos SANTOS, Sao Paulo (BR); Ramon Oliveira VIDAL, Itabuna (BR); Bruno Feres de SOUZA, Sao Luiz (BR); Flavio Mavignier CARCANO, Barretos (BR); Cristovam Scapulatempo NETO, Barretos (BR); Cristiano Ribeiro VIANA, Barretos (BR); Andre Lopes CARVALHO, Barretos (BR)

(73) Assignees: Fleury S/A, Sao Paulo (BR); Hospital Do Cancer Barretos- Fundacao Pio XII, Barretos (BR); Universidade Federal Do Maranhao, Sao Luis (BR)

(21) Appl. No.: 15/117,023

(22) PCT Filed: Nov. 19, 2014
(86) PCT No.: PCT/BR2014/000418

§ 371 Date: Aug. 5, 2016

(30) Foreign Application Priority Data
Feb. 7, 2014 (BR) BR102014003033-6

(51) Int. Cl. C12Q 1/68; G06F 19/20

(52) U.S. Cl. CPC C12Q 1/6886; G06F 19/20; C12Q 2600/112; C12Q 2600/158

(57) ABSTRACT

The present invention refers to a process for classifying tumor samples of unknown and/or uncertain primary origin, specifically including the steps of obtaining patterns of biological activity modulation of tumor of unknown and/or uncertain primary origin and comparing them to an specific and unique group of biomarkers which determine the profiles of biological activity modulation of known origin tumors. The present invention belongs to the molecular biology and genetics field.

### FIELD OF THE INVENTION

The present invention refers to a process for classification of tumor samples of unknown and/or uncertain origin, mainly comprising a step of obtaining biological activity modulation profiles of tumors of unknown and/or uncertain origin and comparison thereof, through a specific and unique group of biomarkers that determines such molecular profiles, with tumors of known origin. The present invention belongs to the field of molecular biology and genetics.

### BACKGROUND OF THE INVENTION

According to the National Cancer Institute of the National Institute of Health (NIH) of the United States, cancer is a term used to designate “diseases in which there is an uncontrolled division of abnormal cells, which have the ability to invade other tissue types.” Other terms such as malignant tumors and neoplasia are also used. According to the World Health Organization (WHO) through its International Agency for Cancer Research (IACR), 4 million cases of cancer are estimated for 2014 and this disease accounts for 8.2 million deaths around the world, in 2012. It is a public health problem with a predicted number of 27 million new cases of cancer for 2030, also in accordance with IARC. The National Cancer Institute of Brazil (MCA) predicts almost 580 new cases of cancer for 2014 and a growing rate of new cases being 20% per year.

Cancer classification is effected in accordance with the organ where it was developed. Lung cancer, for instance, is a classification designating lung as the primary origin of a patient's cancer, also called primary site. About 30% of all tumors tend to spread from their primary origin to other parts of the organism, causing the so-called metastasis or secondary cancer. Classification of a metastatic tumor, such as primary tumors, is also effected in accordance with the organ from which it originated, that is, its primary origin. For example, a metastatic tumor found in the liver but loosened from the intestine is classified as colorectal cancer and not as hepatic cancer because the original organ of this metastatic tumor was the intestine.

Often, a primary tumor cannot be found, there being only possible to find the metastatic tumor. By this way, classification of metastatic tumors in accordance with their primary origin is a vital condition for oncologic patients. Each type of cancer (that is, each primary origin) has its own therapeutic arsenal; therefore, defining the primary origin of a cancer is crucial to allow the oncologist to decide about the treatment.

There is a series of reasons that make it difficult to identify and/or classify the primary origin of a tumor, such as, for example: i) secondary cancer that spreads very fast while primary cancer is too small to be detected; ii) primary cancer was inhibited by the immune system while secondary cancer still goes on growing; iii) secondary cancer has a high degree of cell indifferentiation and exhibits typical tissue architecture.

At present, classification of primary origin of metastatic tumors is made mainly through immunohistopathology examinations. A pathologist analyses a tumor biopsy sample, uses some biomarkers (antibodies), may resort to typical staining tools and then classifies it. Imaging tools has also been of great help in tumor classification, such as mammography, ultrasound, magnetic resonance, X-ray examinations and more recently PET-CT examinations.

Such techniques are capable of classifying 95% of all cancer cases. The great bias in this form of classification is the subjective and dependent character of each pathologist/radiologist experience. Literature has discussed rates of up to 50% of non-agreement in tumor classification between 2 or more physicians who analyze the same sample/patient. Therefore, in 5% of all cancers it is not possible to determine their primary origin; something around 700.000 people in the world per year. With regard to these cases, the “type” of cancer attributed to these patients is the Tumor of Unknown and/or Uncertain Primary Origin (within the International Classification of Diseases (ICD-10), codes C76 to C80).

This uncertainty in the primary origin of a tumor results in a bad prognostic for a patient with an average survival rate of 6 to 9 months only, since there are no definitions of treatment for most patients in this situation. Tumors of Unknown and/or Uncertain Primary Origin are the 8^{th} more frequent and the 4^{th} more lethal type of cancer. Currently, approaches related to this type of cancer mainly focus on understanding the biology directed to metastasis.

Many immunohistochemical markers have been suggested to predict tumor origins. As recently suggested by some scientific papers about this theme, the panel of markers can include cytokeratins (CK7; CK-20), TTF-1; markers of ovary/breast, HEPAR-1, of renal cells, placental alkaline fosfatase/OCT-4, WT-1/PAX-8, synaptophysin and chromogranin. Immunohistochemical markers generally accurately predict the primary origin in 35-40% of precocious metastatic cancers. Currently, most cases are diagnosed from FFPE samples (formalin-fixed, paraffin-embedded samples) derived from biopsy procedures.

Concerning patent literature, some documents refer to classification of tumors, including those of unknown and/or uncertain origin.

U.S. Pat. No. 7,622,260 refers to the use of microarrays and a method of analyzing metastatic cell samples. It further teaches that there should be measured biomarkers associated with at least two types of carcinomas, describing specific groups of markers which should be used in the classification of certain types of cancers. Similarly, WO 2002/103320 refers to methods of diagnosing cancer using a series of genetic markers, wherein the expression level of these biomarkers relates to the data of patients having cancer. US Patent Application 2011/0230357 discloses a method of determining the primary origin of unknown tumors, comprising the step of comparing the expression profile of a sample to a classification parameter, wherein said classifier parameter is specific to a tissue through a proper group of biomarkers. WO 2013/002750 refers to a method of classifying tumors of unknown origin. It describes steps of producing and amplifying specific cDNA molecules having more than 50 transcriptions to compare amplification levels to expression levels of genes in tumors. Said document further mentions a set of 87 mRNA sequences corresponding to tumor-related genes.

By this way, it can be observed that there are documents teachings tumor classification methods. Nevertheless, it can be noted that one of the main differences among them is the group/subgroup of biomarkers which each of these documents discloses, since the choice of determined groups/subgroups of biomarkers will be essential for determining different sensitivities in the identification and classification of tumors. Hence, the difference between the present invention and the method of classifying tumors of unknown and/or uncertain origin taught by the above-mentioned state-of-the-art documents resides in that the present invention comprises a group of 95 biomarkers differing from the group of biomarkers disclosed in said state-of-the-art documents. The method of tumor classification of the present invention comprises a new and inventive group of biomarkers which must be taken in consideration together, and whose combination of genes permits to provide a more efficient and accurate classification method compared to those of the state-of-the-art. Hence, according to the present inventor's opinion, the fact of further comprising a new group of biomarkers not only imparts novelty but also inventive step to the present application, since it would not be obvious for a person skilled in the art to carry out the selection and the presently disclosed combination of biomarkers and even correlate them in the same way as described herein. Hence, in view of the foregoing, one may note that the present state-of-the-ad further lacks technical and functional solutions capable of providing a more precise classification of samples of tumors of unknown and/or uncertain origin, that is, in a more efficient and non-subjective form. Therefore, it can be said that state-of-the art technologies, although particularly useful, do not allow for one to obtain methods of classifying tumors of unknown and/or uncertain origin in an efficient, cost-effective and rapid form as the one provided by the present invention, which is described in detail below.

### OBJECTS OF THE INVENTION

In view of the foregoing, there is a need for development of methods which will help in identification and classification of tumors, mainly those of unknown and/or uncertain origin, which will provide less subjective and more accurate results and higher specificity. Thus, the present invention will solve these and other state-of-the-art problems by presenting a rapid, cost-effective and efficient way of also classifying tumors by means of an alternative and innovative process, which methodology was fully in-house developed, with the proof of principles tested and validated in practice. In this sense, this invention also comprises a new and inventive group of biomarkers which can be used in the classification and ranking of the more probable types of cancers to which a tumor sample could belongs.

The present invention is firstly directed to a genes and data selection system referring to biologic activity modulation in samples of tumors whose known primary site is known such that this information can be subsequently used to make comparisons with data referring to biologic activity modulation of tumor samples of unknown and/or uncertain origin. The genes selection system construction was specifically designed with quality control checkpoints such that only those samples with biological significance for the presently disclosed process are used.

Furthermore, a new, inventive and unique group of biomarkers is also disclosed, this group being essential to generate specific profiles and biological activity modulation patterns for each tumor type, allowing the classification of probable origins of a tumor.

A process for manipulating and purifying tumor biological sample analytes is also disclosed, said process being efficient so that data can be collected concerning tumor samples, which are either of known origin or unknown and/or uncertain origin, wherein these data are compared to the data of the system. After generation and analysis of biological activity modulations profiles of these new biomarkers group presented here in tumor samples of unknown and/or uncertain origin, these data are compared to the data of the system. After this comparison, it is possible to obtain statistic data representing similarity, by means of statistical probability, of each interrogated sample being associated with one or more types of tumors. Preferably, the result is given in a ranking form showing percent chances for each sample to be associated with one or more tumor types. More preferably, the chances of each sample of tumor of unknown and/or uncertain origin being associated with at least three types of tumor are presented. This combination of innovations represents not only economic advantages but also clear technological advances.

Thus, one object of the present invention is to provide a process and apparatus for classification of tumor samples, specifically tumors of unknown and/or uncertain origin, as well as a kit for classification of tumors.

### SUMMARY OF THE INVENTION

By this way, in order to achieve the objects and technical effects related above, the present invention refers to a process for classifying tumor samples of unknown and/or uncertain origin, comprising the steps of:
a) obtaining, from preferably virtual samples of tumors of known origin, the biological activity modulation level of a predetermined group of biomarkers comprising: arf5, batf, c6, ca2, cadps, capn6, ccna1, cdca3, cdh16, cdh17, celsr2, chrm3, cox11, cpeb1, csf2rb, cx3cr1, elac2, elavl4, emx2, eps8l3, ern2, esr1, fgf9, foxa1, foxg1, hlf, hoxa9, hoxc10, hoxd11, hsdl2, htr3a, ibsp, kncj12, kdelr2, kif13a, kif15, kif2c, klhdc8a, ly6d, ly6e, ly6h, map2k6, meis1, nb1a00301, odz1, panx1, pax8, pparg, prame, prdm5, prdm8, prkcq, prkra, pycr1, rax, rgs17, rtdr1, s100pbp, sdc1, selenbp1, slc35f2, slc35f5, slc43al, slc6a1, s1c7a5, sp2, spred2, stc1, tmprss3, tmprss4, traj17, trim15, tshr, tssc4, upk1b, vgll1, vps33b, wwc1, znf365, nkx2-1, bcl11b, sh2d1a, prm1, elfn2, slc45a3, fam167a, gjb6, mls, lamp2, capsl, cyorf15a, c14orf105, gfap, fga and stc2;
b) determining, from preferably real samples of tumors of unknown and/or uncertain origin, the biological activity modulation level of the same predetermined group of biomarkers used in step a);
c) normalizing the biological activity modulation level of biomarkers of a) and b) to obtain the ratio (foldchange) between each discriminating biomarker with each normalizing biomarker;
d) comparing the profiles of the biological activity modulation level of the biomarkers in tumor samples of known origin to the profiles of the biological activity modulation level of biomarkers in tumor samples of unknown and/or uncertain origin, preferably classifying the sample in a ranking form.

Preferably, the samples of tumors of known origin are obtained from analysis or experiments of DNA microarrays or Real-Time PCR.

In a preferred embodiment, types of breast and/or uterus and/or ovary cancer tumors are not used for obtaining profiles of the biological activity modulation level of biomarkers which will be compared to unknown and/or uncertain tumor samples of male patients.

In a preferred embodiment, the prostate cancer tumor type is not used to obtain profiles of the biological activity modulation level of biomarkers which will be compared to unknown and/or uncertain tumor samples of female patients.

The normalization step uses normalizing biomarkers to perform normalization of the biological activity modulation of tumors of known origin and tumors of unknown and/or uncertain origin. Preferably, said normalizing biomarkers are selected from the group comprising the whole group of biomarkers described herein. Preferably, 4 normalizing biomarkers are selected, wherein (1) is arf5, (2) is sp2, (3) is vps33b, and (4) is an additional one selected from the group comprising: kdler2 or /y6e or panx1.

Additionally, in a preferred embodiment, normalization is carried out by obtaining the ratio (foldchange) between the value related to the activity modulation of each discriminating biomarker and the value related to the activity modulation of each normalizing biomarker. Comparison of these data of tumor samples of known origin with the data of tumor samples of unknown and/or uncertain origin is carried out preferably using computational tools. More preferably, techniques presented in Machine Learning (ML) algorithms such as RandomForest (RF) technique—as described by Leo Breiman. 2001. Random Forests. Mach. Learn. 45, 1, 5-32—are used to relate the data of known origin samples to classify tumor samples of unknown and/or uncertain origin.

In a preferred embodiment, the present process for classifying tumor samples of unknown and/or uncertain origin uses as sub-step of a) a quality control process for samples of tumors of unknown and/or uncertain origin to determine whether the biological material and/or results of the analysis of its biological activity modulation have sufficient quality to produce reliable data during analysis thereof.

Said quality control process applied to tumor biological samples of known origin to obtain profiles of biological activity modulation level of biomarkers of tumor samples of known origin in a process for classifying tumor samples. The cited quality control process preferably for virtual biological samples of known origin comprising the steps of:

A. submitting the obtained samples to a pre-selection by the following evaluation criteria:
i. determine if the sample is of origin different from laboratorial or xenotransplant cell lines;
ii. determine if the sample is free of any cancer-related treatment;
iii. determine if the sample is a tumor sample;
iv. determine if the primary origin of the tumor sample is known;
v. determine if the sample is a human (Homo sapiens) sample;

wherein said sample that had all the evaluation criteria questions positively answered is pre-selected to be used as a biological sample of a tumor biological sample of known origin having high quality;

B. selecting once more from the samples selected in a) those samples comprising available data about the following group of biomarkers: arf5, batf, c6, ca2, cadps, capn6, ccna1, cdca3, cdh16, cdh17, celsr2, chrm3, cox11, cpeb1, csf2rb, cx3cr1, etac2, elavl4, emx2, eps8l3, ern2, esr1, fgf9, foxa, foxg1, hlf, hoxa9, hoxc10, hoxd11, hsdl2, htr3a, ibsp, kncj12, kdelr2, kif13a, kif15, kif2c, klhdc8a, ly6d, ly6e, ly6h, map2k6, meis1, nbla00301, odz1, panx1, pax8, pparg, prame, prdm5, prdm8, prkcq, prkra, pycr1, rax, rgs17, rtdr1, s100pbp, sdc1, selenbp1, slc35f2, slc35f5, slc43a1, slc6a1, slc7a5, sp2, spred2, stc1, tmprss3, tmprss4, traj17, trim15, tshr, tssc4, upk1b, vgll1, vps33b, wwc1, znf365, nkx2-1, bcl11b, sh2d1a, prm1, elfn2, slc45a3, fam167a, gjb6, rnls, lamp2, capsl, cyorf15a, c14orf105, gfap, fga and stc2;

C. selecting from the set of biomarkers described in b) at least three biomarkers having low variation coefficients among all the analyzed tumor samples of known origin;

D. using said at least three biomarkers selected from c) as quality control parameter, fulfilling the following relation therebetween:

0.01<[(Biomarker_1+Biomarker_2)/2]/Biomarker_3<10.00;

wherein in case the sample data fall within the range mentioned above, same is selected as being a quality tumor sample of known origin.

Thus, said selected samples can be subjected to a normalization step for the classification of tumor samples of unknown and/or uncertain origin.

In a preferred embodiment, the at least three biomarkers from these quality control comprise ly6e, kdelr2 and panx1.

Said quality control process for preferably real biological samples of unknown and/or uncertain origin comprises the steps of:

I) processing the obtained samples for extraction and purification of the biological material analytes;

II) subjecting said analytes to amplification in which collection of data of the respective amplification cycles (CycleThreshold—Ct) is made;

III) the sample of II) must be submitted to the following evaluation criterion:

Ct 10.00<Ct value of the analyzed biomarker<Ct 40.00;

wherein in case the sample falls within the range mentioned above, same is selected as being a tumor sample having high quality.

Thus, the selected samples can be subjected to normalization steps for classification of the tumor samples of unknown and/or uncertain origin.

In a preferred embodiment, said biomarker(s) used in this quality control can be one or more genes selected from the group comprising: arf5, sp2, vpss33b, tssc4, kdelr2, 1ye6 and panx1.

### BRIEF DESCRIPTION OF THE DRAWINGS

FIG. 1 is a flowchart illustrating an embodiment of the process for generating gene expression profiles of preferably virtual tumor samples of known origin;

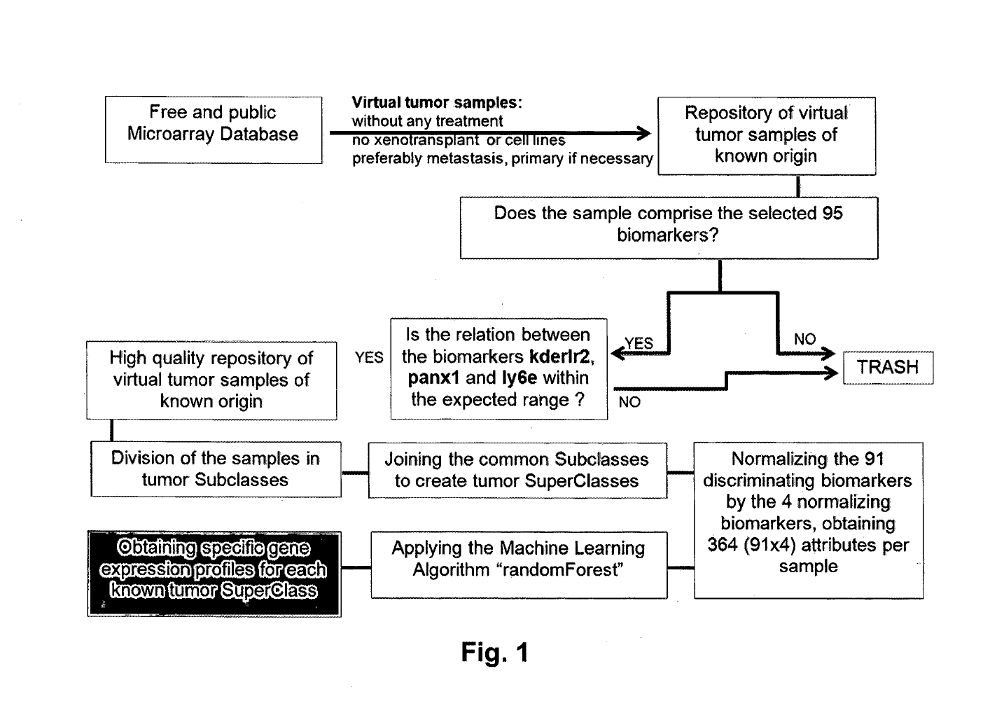

FIG. 2 is a flowchart illustrating an embodiment relative to processing of samples, quality control and generation of gene expression profiles of unknown and/or uncertain, preferably real, tumor samples, to compare with the expression profiles of tumor samples of known origin, for example, those obtained as illustrated in FIG. 1.

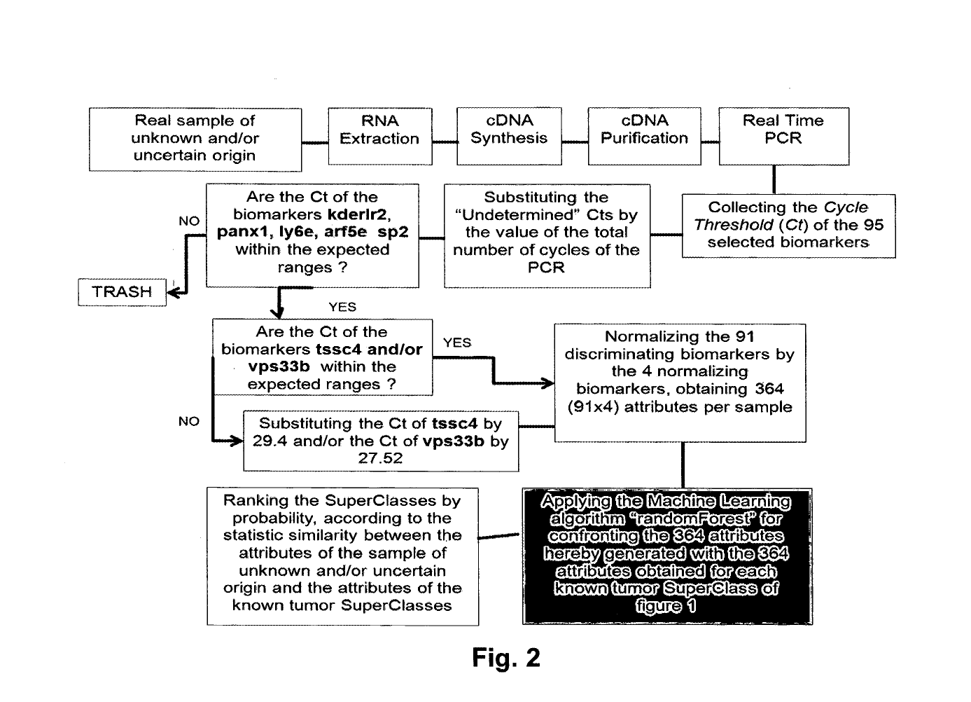

Attention should be drawn to the fact that the flowcharts in both figures filled in gray color disclose an interconnection point between the two flowcharts.

### DETAILED DESCRIPTION OF THE INVENTION

The present invention refers to several details which shall only be interpreted as examples of how the invention is to be applied, and not as limitative of the application thereof.

### Biological Activity Modulation

By the term “biological activity modulation” of the present invention it is meant any quantitative measurement of quantity/expression/regulation of elements, such as, for example, DNA, RNA and/or proteins in biological samples. In a preferred embodiment, said term encompasses quantitatively measurement of gene expression. Several means can be used to verify the gene expression.

### Biological Samples

The “biological samples” of the present invention comprise any parts of living beings, preferably mammals, yet more preferably humans, which can be used to obtain biological information from determined organism and/or organ and/or tissue and/or cell and/or molecule. In the present invention, said biological samples are mainly molecular biological elements (analytes) such as, for example, DNA, RNA and/or proteins, preferably those from primary or metastatic cancer. In the present invention, by the term “real biological samples” it is meant those samples which were experimentally processed, for example, which are subjected to bench tests (wetlab) whereas by the term “virtual biological samples” it is meant those samples which were processed and wherein the data, for example, are available in public databanks and can be gotten for free from the internet or other means.

### Biomarkers

Genes having different functions to compose the group of biomarkers of the present invention were selected. These “biomarkers” comprise any entities which have their physical-chemical-biological parameters measured by analytical and/or scientific instrumentation. In the present invention, the definition of the group of biomarkers is considered to be an improvement in the state-of-the-art since it discloses a novel and inventive group of biomarkers for the classification of tumors of unknown and/or uncertain origin. In a preferred embodiment, the group of biomarkers of the present invention comprises: arf5, batf, c6, ca2, cadps, capn6, ccna1, cdca3, cdh16, cdh17, celsr2, chrm3, cox11, cpeb1, csf2rb, cx3cr1, elac2, elavl4, emx2, eps8l3, ern2, esr1, fgf9, foxa1, foxg1, hlf, hoxa9, hoxc10, hoxd11, hsdl2, htr3a, ibsp, kncj12, kdelr2, kif13a, kif15, kif2c, klhdc8a, ly6d, ly6e, ly6h, map2k6, meis1, nbla00301, odz1, panx1, pax8, pparg, prame, prdm5, prdm8, prkcq, prkra, pycr1, rax, rgs17, rtdr1, s100pbp, sdc1, selenbp1, slc35f2, slc35f5, slc43a1, slc6a1, slc7a5, sp2, spred2, stc1, tmprss3, tmprss4, traj17, trim15, tshr, tssc4, upk1b, vgll1, vps33b, wwc1, znf365, nkx2-1, bcl11b, sh2d1a, prm1, mls, lamp2, c14orf105, gfap, fga,stc2, elfn2, slc45a3, fam167a, gjb6, capsl, and cyorf15a (see Table 1).

TABLE 1

| Gene | | Assay Code used | | | |
|---|---|---|---|---|---|
| (Official | Access Code | in Real-Time PCR | Probeset IDs Codes analyzed in | | |
| Symbol) | (Ref Seq-NCBI) | (Life Technologies) | microarray files (Affymetrix) | | |
| ARF5 | NM_001662.3 | Hs01018622_m1 | 201526_at | | |
| BATF | NM_006399.3 | Hs00232390_m1 | 205965_at | | |
| BCL11B | NM_022898.1 | Hs01102259_m1 | 219528_s_at | | |
| C14orf105 | NM_018168.2 | Hs00216847_m1 | 220084_at | | |
| C6 | NM_000065.2 | Hs00163840_m1 | 210168_at | | |
| CA2 | NM_000067.2 | Hs01070108_m1 | 209301_at | | |
| CADPS | NM_003716.3 | Hs00186598_m1 | 204814_at | | |
| CAPN6 | NM_014289.3 | Hs00560073_m1 | 202965_s_at | 202966_at | |
| CAPSL | NM_001042625.1 | Hs00376162_m1 | 236085_at | | |
| CCNA1 | NM_003914.3 | Hs00171105_m1 | 205899_at | | |
| CDCA3 | NM_031299.4 | Hs00229905_m1 | 221436_s_at | | |
| CDH16 | NM_004062.3 | Hs00187880_m1 | 206517_at | | |
| CDH17 | NM_004063.3 | Hs00184865_m1 | 209847_at | | |
| CELSR2 | NM_001408.2 | Hs00154903_m1 | 204029_at | 36499_at | |
| CHRM3 | NM_000740.2 | Hs00265216_s1 | 214596_at | | |
| COX11 | NR_027942.1 | Hs00362087_m1 | 211727_s_at | 214277_at | 203551_s_at |
| CPEB1 | NM_001079535.1 | Hs00229015_m1 | 219578_s_at | | |
| CSF2RB | NM_000395.2 | Hs00166144_m1 | 205159_at | | |
| CX3CR1 | NM_001337.3 | Hs00365842_m1 | 205898_at | | |
| CYorf15A | NR_045129.1 | Hs00416710_m1 | 232618_at | 236694_at | |
| ELAC2 | NM_018127.6 | Hs01004288_m1 | 201767_s_at | 201766_at | |
| ELAVL4 | NM_001144776.1 | Hs00222634_m1 | 206051_at | | |
| ELFN2 | NM_052906.3 | Hs00287464_s1 | 1559072_a_at | 1563108_at | 1560713_a_at |
| EMX2 | NM_004098.3 | Hs00244574_m1 | 221950_at | | |
| EPS8L3 | NM_024526.3 | Hs00225968_m1 | 219404_at | | |
| ERN2 | NM_033266.3 | Hs01086607_m1 | 214372_x_at | | |
| ESR1 | NM_000125.3 | Hs00174860_m1 | 211233_x_at | 215551_at | 211234_x_at |
| FAM167A | NM_053279.2 | Hs00697562_m1 | 226614_s_at | 233641_s_at | |
| FGA | NM_000508.3 | Hs00241029_m1 | 205650_s_at | 205649_s_at | |
| FGF9 | NM_002010.2 | Hs00181829_m1 | 206404_at | | |
| FOXA1 | NM_004496.3 | Hs04187555_m1 | 204667_at | | |
| FOXG1 | NM_005249.4 | Hs01850784_s1 | 206018_at | | |
| GFAP | NM_002055.4 | Hs00909236_m1 | 203539_s_at | 203540_at | |
| GJB6 | NM_006783.4 | Hs00272726_s1 | 231771_at | | |
| HLF | NM_002126.4 | Hs00171406_m1 | 204753_s_at | 204755_x_at | 204754_at |
| HOXA9 | NR_037940.1 | Hs00365956_m1 | 209905_at | 214651_s_at | |
| HOXC10 | NM_017409.3 | Hs00213579_m1 | 218959_at | | |
| HOXD11 | NM_021192.2 | Hs00360798_m1 | 214604_at | | |
| HSDL2 | NM_001195822.1 | Hs00953689_m1 | 209512_at | 209513_s_at | 215436_at |
| HTR3A | NR_046363.1 | Hs00168375_m1 | 216615_s_at | 217002_s_at | |
| IBSP | NM_004967.3 | Hs00173720_m1 | 207370_at | | |
| KCNJ12 | NM_021012.4 | Hs00253248_s1 | 208567_s_at | 207110_at | 208566_at |
| KDELR2 | NM_006854.3 | Hs00199277_m1 | 200700_s_at | 200699_at | 200698_at |
| KIF13A | NM_001105568.2 | Hs00223154_m1 | 220777_at | | |
| KIF15 | NM_020242.2 | Hs00173349_m1 | 219306_at | | |
| KIF2C | NM_006845.3 | Hs00901710_m1 | 209408_at | 211519_s_at | |
| KLHDC8A | NM_018203.1 | Hs00217063_m1 | 219331_s_at | | |
| LAMP2 | NM_002294.2 | Hs00174481_m1 | 200821_at | 203042_at | 203041_s_at |
| LY6D | NM_003695.2 | Hs00170353_m1 | 206276_at | | |
| LY6E | NM_002346.2 | Hs00158942_m1 | 202145_at | | |
| LY6H | NM_001135655.1 | Hs01108584_m1 | 206773_at | | |
| MAP2K6 | NM_002758.3 | Hs00992389_m1 | 205698_s_at | 205699_at | |
| MEIS1 | NM_002398.2 | Hs00180020_m1 | 204069_at | | |
| NBLA00301 | NC_000004.11 | Hs00257335_s1 | 219791_s_at | | |
| NKX2-1 | NM_003317.3 | Hs00163037_m1 | 211024_s_at | 210673_x_at | |
| ODZ1 | NM_001163278.1 | Hs00173872_m1 | 205728_at | | |
| PANX1 | NM_015368.3 | Hs00209790_m1 | 204715_at | | |
| PAX8 | NM_013953.3 | Hs01015249_m1 | 221990_at | 207923_x_at | 214528_s_at |
| PPARG | NM_015869.4 | Hs01115513_m1 | 208510_s_at | | |
| PRAME | NM_206956.1 | Hs01022301_m1 | 204086_at | | |
| PRDM5 | NM_018699.2 | Hs00924602_m1 | 220792_at | | |
| PRDM8 | NM_020226.3 | Hs01027634_g1 | 219835_at | | |
| PRKCQ | NM_001242413.1 | Hs00989970_m1 | 210038_at | 210039_s_at | |
| PRKRA | NM_001139518.1 | Hs00269379_m1 | 209139_s_at | | |
| PRM1 | NM_002761.2 | Hs00358158_g1 | 206358_at | | |
| PYCR1 | NM_153824.1 | Hs01048016_m1 | 202148_s_at | | |
| RAX | NM_013435.2 | Hs00429459_m1 | 208242_at | | |
| RGS17 | NM_012419.4 | Hs00202720_m1 | 220334_at | | |
| RNLS | NM_018363.3 | Hs00218018_m1 | 220564_at | | |
| RTDR1 | NM_014433.2 | Hs02330211_m1 | 220105_at | | |
| S100PBP | NM_001256121.1 | Hs00224254_m1 | 218370_s_at | | |
| SDC1 | NM_002997.4 | Hs00896423_m1 | 201286_at | 201287_s_at | |
| SELENBP1 | NM_001258288.1 | Hs00259932_m1 | 214433_s_at | | |
| SH2D1A | NM_001114937.2 | Hs00158978_m1 | 211210_x_at | 211211_x_at | 210116_at |
| SLC35F2 | NM_017515.4 | Hs00213850_m1 | 218826_at | | |
| SLC35F5 | NM_025181.2 | Hs00228615_m1 | 220123_at | | |
| SLC43A1 | NM_003627.5 | Hs00992327_m1 | 204394_at | | |
| SLC45A3 | NM_033102.2 | Hs00263832_m1 | 228696_at | 238499_at | |
| SLC6A1 | NM_003042.3 | Hs01104469_m1 | 205152_at | | |
| SLC7A5 | NM_003486.5 | Hs01001183_m1 | 201195_s_at | | |
| SP2 | NM_003110.5 | Hs00370726_m1 | 204367_at | | |
| SPRED2 | NM_001128210.1 | Hs00986220_m1 | 212466_at | 214026_s_at | 212458_at |
| STC1 | NM_003155.2 | Hs00174970_m1 | 204595_s_at | 204596_s_at | 204597_x_at |
| STC2 | NM_003714.2 | Hs00175027_m1 | 203439_s_at | 203438_at | |
| TMPRSS3 | NM_032404.2 | Hs00225161_m1 | 220177_s_at | | |
| TMPRSS4 | NM_001173551.1 | Hs00854071_mH | 218960_at | | |
| TRAJ17 | NC_000014.8 | Hs00413014_g1 | 217412_at | | |
| TRIM15 | NM_033229.2 | Hs00264400_m1 | 36742_at | 210885_s_at | 210177_at |
| TSHR | NM_000369.2 | Hs01053846_m1 | 215442_s_at | 210055_at | 215443_at |
| TSSC4 | NM_005706.2 | Hs00185082_m1 | 218612_s_at | | |
| UPK1B | NM_006952.3 | Hs00199583_m1 | 210064_s_at | 210065_s_at | |
| VGLL1 | NM_016267.3 | Hs00212387_m1 | 215729_s_at | 215730_at | 205487_s_at |
| VPS33B | NM_018668.3 | Hs00218719_m1 | 218415_at | 44111_at | |
| WWC1 | NM_015238.2 | Hs00392086_m1 | 213085_s_at | 216074_x_at | |
| ZNF365 | NM_014951.2 | Hs00209000_m1 | 206448_at | | |

In some occasions, some biomarkers were selected to be used, for example, as basis for calculation of quality control parameters or as sample normalizers. Preferably, biomarkers used as basis for calculation of quality control parameters or as sample normalizers are selected from the group consisting of: arf5, sp2, vpss33b, tssc4, kdelr2, lye6, and panx1. In the case of biomarkers for normalization of data of tumor samples of known origin or of unknown and/or uncertain origin, 4 biomarkers are preferably used: (1) is arf5, (2) is sp2, (3) is vps33b, and (4) is one selected from the group comprising: kdler2 or ly6e or panx1. With regard to biomarkers used as quality control for selecting samples of known origin, preferably virtual samples of high quality, ly6e, kdelr2 and panx1 are preferably used. In the case of the biomarkers used as quality control for selection of samples of unknown and/or uncertain origin, preferably real samples of high quality, at least one biomarker of the group comprising arf5, sp2, vpss33b, tssc4, kdelr2, lye6, and panx1 is preferably used.

### Tumors of Known/Unknown Origin

Primary or metastatic primary tumors may not have their origin defined, leading the patient to suffer from a cancer of unknown and/or uncertain origin. The expression “tumor of unknown and/or uncertain origin” can be interchangeably substituted by the expression “tumor of primary and/or metastatic unknown and/or uncertain origin” or the like, in the present invention without compromising same.

The expressions “tumor of known origin” or “tumor sample of known origin” used in the present invention correspond to tumor wherein it was possible to determine its primary origin and, consequently, it was possible to establish from which tissue/organ the tumor originates.

With regard to the process for classifying tumor samples of unknown and/or uncertain origin, it comprises the step a) of obtaining from preferably virtual samples the biological activity modulation level of a predetermined group of biomarkers comprising: arf5, batf, c6, ca2, cadps, capn6, ccna1, cdca3, cdh16, cdh17, celsr2, chrm3, cox11, cpeb1, csf2rb, cx3cr1, elac2, elavl4, emx2, eps8l3, ern2, esr1, fgf9, foxa1, foxg1, hlf, hoxa9, hoxc10, hoxd11, hsdl2, htr3a, ibsp, kncj12, kdelr2, kif13a, kif15, kif2c, klhdc8a, ly6d, ly6e, ly6h, map2k6, meis1, nbla00301, odz1, panx1, pax8, pparg, prame, prdm5, prdm8, prkcq, prkra, pycr1, rax, rgs17, rtdr1, s100pbp, sdc1, selenbp1, slc35f2, slc35f5, slc43a1, slc6a1, slc7a5, sp2, spred2, stc1, tmprss3, tmprss4, traj17, trim15, tshr, tssc4, upk1b, vgll1, vps33b, wwc1, znf365, nkx2-1, bcl11b, sh2d1a, prm1, elfn2, s1c45a3,fam167a, gjb 6, mls, lamp2, capsl, cyorf15a, c14orf105, gfap, fga and stc2; wherein, for example, the obtainment from preferably virtual samples tumors of known origin comprises building a repository of files with data, preferably of gene expression based on platforms of DNA microarrays obtained and available online in the platform Array Express of EMBL-EBI (www.ebi.ac.uk/arrayexpress), categorized according to Table 2.

In this public and free platform many (raw and processed) files are available, which comprise several data about biological activity modulation of biological samples, including tumor samples; said platform is constantly updated and files and information are available to the public.

TABLE 2

| Tumor super- | | |
|---|---|---|
| classes | Subclass(es) composing it | Access Code (ArrayExpress) |
| Adrenal | Adrenocortical Carcinoma | E-GEOD 2109, E-GEOD 33371, E-TABM 311, |
| | | E-GEOD 19750 |
| Breast | Ductal Carcinoma | E-GEOD 2109, E-GEOD 5460, |
| | Inflammatory Carcinoma | E-TABM 185, E-GEOD 5847, |
| | Lobular Carcinoma | E-GEOD 1006 |
| Gastroesophageal | Esophagus Adenocarcinoma | E-GEOD 2109, GSE15459, E-GEOD 22377, |
| | Stomach Adenocarcinoma | E-GEOD 26886, E-GEOD 37203, E-GEOD |
| | | 1420, E-GEOD 29272 |
| Nonseminomatous | Mixed Germinative Cells | E-GEOD 2109, E-GEOD-18155, E- |
| Germinative Cells | Yolk Sac Cells | GEOD 3218, E-GEOD 10615, E- |
| | Testicular/Ovarian Teratoma | TABM 185 |
| Seminomatous | Seminoma/Dysgerminoma | |
| Germinative Cells | | |
| Gastrointestinal | Gastrointestinal Stromal Cells | E-GEOD 20708, E-GEOD 17743, E-GEOD |
| Stromal Tumor | | 8167 |
| Head and Neck | Adenoid Cystic Carcinoma - Salivary | E-GEOD 28996 |
| (Salivary Gland) | Gland | |
| Intestine | Colorectal Adenocarcinoma | GSE14333, GSE20916, E-GEOD 4459 |
| Kidney | Oncocytoma | E-GEOD 2109, E-GEOD 15641, |
| | Renal Cell Carcinoma - Clear Cells | E-GEOD 12090, E-GEOD 19982, |
| | Renal Cell Carcinoma - Chromophobe | E-GEOD 2748 |
| | Renal Cell Carcinoma - Papillary | |
| Liver | Hepatocellular Carcinoma | |
| Lung- | Lung Adenocarcinoma | E-GEOD 2109, GSE14520, G5E9829, E- |
| Adenocarcinoma/ | Large Cell Carcinoma/ | GEOD 6465, E-TABM 36 |
| Large Cell Carcinoma | Bronchoalveolar | |
| Lung-Small | Small Cell Carcinoma | E-GEOD 15240, E-GEOD 20189, E-GEOD |
| Cell Carcinoma | | 43346, E-GEOD 302019, E-GEOD3141 |
| Lymphoma | Hodgkin | E-GEOD 2109, E-GEOD 10524, |
| | Diffuse Large B cells | E-GEOD 34339, E-GEOD 19246, |
| | Peripheral T Cells | E-GEOD 17920, E-GEOD 12453, E-GEOD |
| | | 12453, E-GEOD 19069, E-GEOD 19069, E- |
| | | GEOD 6338, E-GEOD 34171 |
| Melanoma | Uveal | E-GEOD 2109, E-GEOD 19234, E-GEOD |
| | Non-Uveal | 22138, E-GEOD 27831, E-GEOD 7553, E- |
| | | GEOD 3189 |
| Mesothelioma | Mesothelioma | E-GEOD 29211, E-GEOD 12345, E-GEOD |
| | | 2549 |
| Neuroendocrine | Pheochromocytoma/Paraganglioma | E-MTAB 733, E-GEOD 2841, |
| Tumors | Lung - Carcinoid | E-GEOD 39612 |
| | Merkel Cell Carcinoma | |
| Ovary | Clear Cell Adenocarcinoma | E-GEOD 2109, E-GEOD 29460, |
| | Endometrioid Adenocarcinoma | E-GEOD 6008, E-GEOD 9899, |
| | Mucinous Adenocarcinoma | E-GEOD 18520 |
| | Serous Papillary Adenocarcinoma | |
| | Serous Adenocarcinoma | |
| | Serous or Serous Papillary Carcinoma | |
| Pancreas | Pancreatic Ductal Carcinoma | E-GEOD 32688, E-GEOD 22780, E-MEXP |
| | Cholangiocarcinoma | 1121, E-MEXP 950, E-MEXP 2780, E-GEOD |
| | | 19281, E-GEOD 32676, E-GEOD 2109, E- |
| | | GEOD 34166, E-GEOD 15765 |
| Prostate | Prostate Adenocarcinoma | E-GEOD 2109, E-GEOD 17951 |
| Sarcoma | Chondrosarcoma | E-GEOD 2109, E-GEOD 21122, E-GEOD |
| | Lelomyosarcoma | 30929, GSE14325, E-GEOD 32375, |
| | Liposarcoma/MyxoidLiposarcoma | GSE12865, E-GEOD 16088, E-GEOD |
| | Fibrous Malignant Histiocytoma/ | 16091, E-GEOD 37562, E-GEOD 17679, E- |
| | Myxofibrosarcoma | GEOD 34620, E-GEOD 6481, E-MEXP 353, E- |
| | Bi or Monophasic Synovial Sarcoma | GEOD 21050, E-GEOD 2719, E-TABM 185, E- |
| | Osteosarcoma | GEOD 21222 |
| | Ewing's sarcoma or Primitive | |
| | Neuroectodermal Tumor | |
| Squamous Cell | Uterine Cervix | E-GEOD 2109, E-GEOD 7803, E-GEOD 2109, |
| Carcinoma | Lung | GSE28571, E-GEOD 10245, E-GEOD 3141, E- |
| | Head and Neck/Skin | GEOD 2109, GSE30784, E-GEOD 23036, E- |
| | Esophagus | TABM 185, GSE20347, GSE29001, E-GEOD |
| | | 26886 |
| Thymus | Thymoma | E-GEOD 29695 |
| Thyroid | Follicular Carcinoma | GSE15045, E-GEOD 27155, E-GEOD 2109, |
| | Papillary Carcinoma | E-GEOD 27155, E-TABM 185, E-MEXP 97, E- |
| | Anaplastic carcinoma or Hurthle Cell | MEXP 2442, E-GEOD 6004 |
| | Carcinoma | |
| Urinary | Transitional Cell Carcinoma | E-GEOD 31684, E-GEOD 24152, E-GEOD |
| | Urothelial adenocarcinoma | 3167, E-MEXP 1220, E-GEOD 2109 |
| Uterus | Cervical Adenocarcinoma | E-GEOD 6791, E-GEOD 2109, E-GEOD |
| | Endometrium Carcinoma | 5787, E-GEOD 17025 |

In view of type of available information and the quality of sample, files of the following microarray platforms were used:

A-AFFY-33-AffymetrixGeneChip Human Genome HG-U133A [HG-U133A/B]

A-AFFY-37-AffymetrixGeneChip Human Genome U133A 2.0 [HG-U133A_2]

A-AFFY-44-AffymetrixGeneChip Human Genome U133 Plus 2.0 [HG-U133_Plus_2]

All platforms and samples used in this repository of files were carefully selected, which permitted to obtain data with quality and accuracy higher than those which have not undergone any previous analysis.

Preferably, the selected tumor biological samples of known origin, preferably virtual samples, were subjected to criteria of sample inclusion and quality, i.e. to the claimed quality control process in order to determine whether the biological material and/or results of the analysis of its biological activity modulation have sufficient quality to produce reliable data during analysis thereof. Such quality control process including the following steps:

A. Subject the obtained samples to a pre-selection according to the following criteria of evaluation:

i. determine if the sample is of origin different from laboratorial or xenotransplant cell lines;

ii. determine if the sample is free of any treatment related to cancer;

iii. determine if the sample is a tumor sample;

iv. determine if the primary origin of the tumor sample is known;

v. determine if the sample is a human (Homo sapiens) sample.

wherein the sample that had all evaluation criteria questions answered positively is pre-selected to be use as a tumor biological sample of known origin, having high quality.

Due to the fact that only samples with the characteristics above have been selected, then only data of samples of primary or metastatic human tumors with no treatment are used, which further helps in the classification of tumor samples of unknown and/or uncertain origin and approximates the classification process to the patient's clinical reality.

Table 2, column 3, shows examples of access numbers of the platforms which are useful for obtaining samples and their correspondence with each super-class and subclass of tumor tissue. From these arrangements, taking into account the criteria listed above, as a whole, more than 7,000 samples were used to compose the repository of virtual tumor samples of known origin are selected.

In step B, all obtained files of sample that were in agreement with the criteria of inclusion specified above are subjected to an additional selection to determine the presence of a group of 95 predetermined biomarkers, which were carefully selected based on experimental data which indicates the efficiency of this group in the classification of tumors of unknown and/or uncertain origin.

Next, in step C, at least three biomarkers having low variation coefficients among all the analyzed tumor samples, preferably virtual samples, are selected from the group of biomarkers of step B.

By this way, it was observed that there was an ideal mathematical relation between the samples to determine the quality of the samples on the basis of these biomarkers which show a slight variation in the biological activity modulation, even when analyzed in different tumor super classes in C, as quality control parameter, satisfying the following relation therebetween:

0.01<[(Biomarker_1+Biomarker_2)/2]/Biomarker_3<10.00;

where in case the sample data fall within the range indicated above, the sample is selected as being a tumor sample of known origin, preferably virtual sample, with high quality.

Specifically, biomarkers used in the equation above should be different from each other. More preferably, the samples should satisfy the following condition:

0.01<[(Biomarker_1+Biomarker_2)/2]/Biomarker_3<8.2;

0.07<[(Biomarker_1+Biomarker_3)/2]/Biomarker_2<1.5;

0.61<[(Biomarker_2+Biornarker_3)/2]/Biomarker_1<8.85;

More preferably, the samples shall consider that the biomarkers were selected from the group comprising: ly6e, panx1, and kdelr2. And more specifically and in a non-limitative way, there have been used as biomarkers the following AffymetrixProbeset_IDs representing, and corresponding to, the biomarkers: ly6e, panx1, kdelr2: 202145_at, 200700_s_at and 204715_at.

For the purpose of the present invention, it is understood as high quality sample any sample that has fulfilled the criteria defined in steps A. to D, above.

By way of example, more than 7,000 samples of the repository of files of virtual tumor samples of known origin were reduced to 4.429 samples divided into 25 Super Classes comprising 58 subclasses (Table 2, columns 1 and 2).

Information contained in this data repository will be subsequently used for classifying possible tumor origins, more specifically, the possible origin tissues/organs of real samples from tumors of unknown and/or uncertain origin.

With regard to step b) of the process for classifying tumor samples of unknown and/or uncertain origin, it is determined from preferably real samples of tumors of unknown and/or uncertain origin, the biological activity modulation level of the same predetermined group of 95 biomarkers used in step a).

By way of non-limitative information, the samples tested in this invention were mainly obtained from FFPE (Formalin-fixed, paraffin embedded) preservation samples. Nevertheless, two other preservation forms such as cryopreservation and even the use of fresh, recently biopsied samples can be used.

In order to prepare a sample for RNA extraction, 2 up to 6 cuts having a thickness of approximately 10 micrometers each are ideally used, placed on glass slides (from paraffin block), where one of said slides will be routinely stained with H&E (Hematoxylin & Eosin) pattern and the remaining slides will not be stained.

The tumor region must be delimited, preferably by a pathologist, on the H&E stained slide to avoid that non-tumor tissue is analyzed. Next, said delimited region is used as guide to collect non-stained slides (this can be done using laser microdissection, with no damage) and the obtained material is transferred to a xylol-containing tube.

RNA extraction is then carried out, wherein use of a commercial kit, e.g. RecoverAll™ Total NucleicAcidlsolation Kit for FFPE (Ambion®—Cat. Num. AM 1975) can be used. At the end of the extraction process, RNA is eluted in water free of D/RNAses.

When necessary, cDNA synthesis is conducted by total amplification of transcriptoma, for example, using TransPlexWholeTranscriptomeAmplification Kit (Sigma®—Cat. Num WTA2-10RXN). After the synthesis is complete, cDNA can be purified, for example, with the help of QIAquick PCR Purification Kit* (QIAGEN®—Cat. Num 28104).

To assess the biological activity modulation of biomarkers in tumor samples of unknown and/or uncertain origin, Real-Time PCR is used. For example, all 95 biomarkers have their TaqMan® assays (pair of specific primers and probe FAM-NFQMGB, predesigned in format of inventoried and/or made-to-order by the manufacturer) spotted in lyophilized form in Low Density Array customized by Life Technologies (TLDA Cards—TaqMan®LowDensityArray—Cat. Num. 4342259). Mastermix buffer mixed to cDNA and added to TLDA cards can be, for example, the TaqMan® Gene Expression Master Mix (Life Technologies—Cat. Num. 4369016). Cycling program of reaction in Real-Time PCR equipment with TLDA Card carries out 40 to 60 cycles, preferably 50 cycles.

After cycling, Ct (Cycle Threshold) data are collected using a fixed threshold value of 0.01 to 0.10, preferably 0.05. All biomarkers which do not present amplification and which are marked by the equipment as “Undetermined”, arbitrarily receive a Ct value equal to the number of cycles used, since the expression of this biomarker is practically null.

In order that the sample is considered as having quality sufficient to be analyzed, Ct of some biomarkers is evaluated as shown below:

Ct 10.00<Ct value of the Biomarkers<Ct 40.00

Preferably, specific ranges and specific biomarkers were used to determine a tumor sample quality as can be seen below:

1) Ct 18.00<ARF5<Ct 25.52;

2) Ct 15.63<SP2<Ct 31.63;

3) Ct 16.48<KDELR2<Ct25.53;

4) Ct 19.58<LYE6<Ct29.34;

5) Ct 18.16<PANX1<Ct 27.46;

wherein if the sample does not fall within any of the ranges above, it will not be analyzed.

With regard to those samples selected by the criteria above, Ct values for biomarkers vps33b and tssc4 will be determined as below:

6) Ct24.37<VPS33B<Ct 35.76—only if outside the range, replace by Ct27.52;

7) Ct 25.53<TSSC4<Ct34.90—only if outside the range, replace by Ct29.40.

If a sample passes all criteria, above, after edited where necessary, it is selected as a biological sample of unknown and/or uncertain origin having high quality. Hence, biological samples of high quality are selected to follow the process for classifying tumor samples of unknown and/or uncertain origin.

For the purpose of the present invention, it is understood that a sample of high quality is any sample that has fulfilled the 7 criteria defined above.

By way of example, after application of the above-described quality control process to biological samples of unknown and/or uncertain origin, out of 112 metastatic tumor samples, only 105 samples were selected, whose primary origin was previously independently determined by the consensus of two pathologists, for the carrying out of blind tests to prove concepts and validating the developed methodology.

In step c), the biological activity modulation level of the biomarkers of a) and b) is normalized, wherein a ratio (foldchange) between each discriminating biomarker with each normalizing biomarker is obtained. Preferably, the normalizing biomarkers are obtained from the group comprising an entire group of 95 biomarkers described herein. Priority is given to the selection of 4 normalizing biomarkers of a group comprising (1) arf5, (2) sp2, (3) vps33b and (4) this biomarker is one selected from the group: kdelr2 or ly6e or panx1, wherein the remaining 91 biomarkers were considered discriminating biomarkers.

In the present invention, normalization is carried out either in known tumor samples or unknown and/or uncertain tumor samples. In the case of samples derived from DNA microarrays, data refer to fluorescence intensity, while in the case of samples derived from Real-Time PCT, data refer to amplification cycles that exceed the fixed cycle threshold (Cycle Threshold—Ct), i.e. amplification level reached by each biomarker in the sample through Real-Time PCR. Hence, considering, for example, the total group of 95 biomarkers wherein 91 are discriminating biomarkers and 4 are normalizing biomarkers, there will amount to 364 (91×4) attributes normalized for a sample analyzed by the present invention.

In a preferred embodiment, unknown and/or uncertain tumor samples of male patients are neither analyzed nor compared to samples of breast, ovary and uterus cancers. Illustratively, in this context, the unknown and/or uncertain samples of male patients were compared to 3602 normalized known tumor samples divided into 22 tumor super classes, which composition was obtained from 45 subclasses. In the case of unknown and/or uncertain samples of female patients, samples were neither analyzed nor compared to prostate cancer samples. In this same context, the unknown and/or uncertain samples of female patients were compared to 4300 normalized known tumor samples divided into 24 tumor super classes, which composition was obtained from 57 subclasses.

Finally, step d) makes a comparison between the normalized profiles of the biological activity modulation level of biomarkers in tumor samples of unknown and/or uncertain origin with super classes obtained from normalized profiles of the biological activity modulation level of biomarkers of tumor samples of known origin, wherein the sample is preferably classified in ranking form.

Such classification is basically carried out to determine a similarity degree, based on statistic probability, between the normalized profiles of the biological activity level of biomarkers in tumor samples of unknown and/or uncertain origin with super classes obtained from normalized profiles of the biological activity modulation level of biomarkers of tumor samples of known origin. In this sense, in a preferred embodiment, comparison between the data of tumor sample of known origin and the data of normalized tumor samples of unknown and/or uncertain origin is carried out using computational tools of Machine Learning. More preferably, it is used “Random Forest” tool that operates forming a decision tree committee to relate the data of tumor samples of known origin to the unknown and/or uncertain tumor samples and classify/rank them. More preferably, implementation of RandomForest (RF) package is used in the statistic analysis. The most significant RF parameters are the number of decision trees (ntree), the amount of attributes used in the construction of trees (mtry=sqrt) and the amount of trees (nodesize). These parameters were used, preferably, with the following parameters values: ntree=50, mtry=sqrt(364) and nodesize=1.

Aiming, at illustratively, determining the discriminating capacity of the obtained repository, it is used as evaluation parameter a compilation of results in a confusion matrix (Table of Contingency—Table 3) from a 10-fold Cross Validation used for generating gene expression profiles of each tumor super class, wherein a tumor sample of known origin was considered correctly classified when its classification was the same previously known. The central diagonal line indicates the amounts of samples which were correctly classified.

Further for illustrative purpose only, it was determined the accuracy of the process for classifying tumor samples of unknown and/or uncertain origin, also using a confusion matrix (Table of Contingency—Table 4) as evaluation parameter by compiling the results obtained from 105 real metastatic tumor samples of unknown origin, in blind test format. In this case, the sample was considered correctly classified when its classification was included among the 3 first superclasses of higher statistic probability. The central diagonal line indicates the amount of correctly classified samples.

Additionally, general parameters observed in those 105 real metastatic samples subjected to classification using the process disclose herein (Table 5) were presented. The methodology was capable of correctly classifying more than 80% of the samples.

TABLE 5

| | | Correctly | Incorrectly | |
|---|---|---|---|---|
| | | Classified | Classified | |
| | | Samples: 88 | Samples: 17 | All Samples: 105 |
| Parameters | | (83.80%) | (16.20%) | (100%) |
| Organ | Liver | 10 (11.36%) | 6 (35.29%) | 16 (15.24%) |
| affectedbymetastasis | Lymph node | 64 (72.72%) | 5 (29.41%) | 69 (65.71%) |
| | Lung | 14 (15.90%) | 3 (17.64%) | 17 (16.19%) |
| Gender | Female | 44 (50.00%) | 7 (41.17%) | 51 (48.57%) |
| | Male | 44 (50.00%) | 10 (58.83%) | 54 (51.43%) |
| Number of 10 μM | Average | 3.1 | 3 | 3.05 |
| FFPE Slides | | | | |
| RNA (quality | 260/280 nm | 1.99 | 2.09 | 2.04 |
| and quantity) | 260/230 nm | 1.34 | 1.38 | 1.36 |
| | [μg/uL] | 168.38 | 144.76 | 166.67 |
| | Bioanalyzer | 2.31 | 2.23 | 2.27 |
| | RIN | | | |
| cDNA (quality | 260/280 nm | 1.74 | 1.74 | 1.74 |
| and quantity) | 260/230 nm | 2.38 | 2.38 | 2.38 |
| | [ng/uL] | 917.12 | 899.66 | 908.39 |
| Non-amplified | Average | 34.5 | 34.06 | 34.28 |
| genes | | | | |
| (Real-Time | | | | |
| PCR) | | | | |
| Normalizing | AllAmplified | 62 (70.45%) | 9 (52.94%) | 71 (67.62%) |
| biomarkers | At least one | 26 (29.55%) | 8 (47.06%) | 34 (32.38%) |
| | non-amplified | | | |
| Ranking | 1st place | 59 (67.04%) | — | 59 (56.19%) |
| Position | 2nd place | 22 (25.00%) | — | 22 (20.95%) |
| | 3rd place | 7 (7.95%) | — | 7 (6.67%) |
| | 4th or 5th | — | 4 (23.52%) | 4 (3.81%) |
| | place | | | |
| | 6th to 9th | — | 6 (35.29%) | 6 (5.71%) |
| | place | | | |
| | 10th to 19th | — | 7 (41.17%) | 7 (6.67%) |
| | place | | | |

RIN = RNA Integrity Number provided by Bioanalyzer (Agilent Technologies).

It should be pointed out that the process for classifying tumor samples of unknown and/or uncertain origin, described and illustrated in the present invention, renders as a final result a classification preferably in ranking format, based on the similarity between the interrogated sample and the super classes of tumors of known origin from statistic probabilities. These data do not substitute results obtained by other tests, examinations and anamnesis to which an oncologic patient was or will be submitted. These data are recommended to be used in a complementary way to data already collected or to be collected by the oncologist responsible for each patient. By this way, the results obtained by the present invention are not sufficient to, separately, define the primary origin of a tumor of unknown and/or uncertain origin.

The present invention further comprises an apparatus/system for classifying primary or metastatic tumor samples of unknown and/or uncertain origin, involving means for conducting the process for classifying tumor samples of unknown and/or uncertain origin, disclosed herein. In a preferred embodiment, the apparatus of the present invention may comprise electronic means (computers, hardwares, softwares) capable of processing information generated and analyzed by the process for classifying tumor samples of unknown and/or uncertain origin.

Additionally, the present invention refers to a kit for classification of tumor samples of unknown and/or uncertain origin. In a preferred embodiment, said kit comprises means for detecting expression levels of one or more biomarkers of the present invention. Optionally, the kit comprises reagents which specifically bind to the biomarkers listed herein such as, for example, nucleotide probes. Additionally, said kit can further comprise electronic devices for processing information about biological activity modulation such that the kit can produce date referring to similarity of the sample to each tumor super class.

The present invention further comprises using 11 determined biomarkers: cdh16, fga, gfap, kcnj12, nkx2-1, prm1, tshr, elfn2, lamp2, stc1, stc2 and at least one of arf5, batf, bcl11b, c14orf105, c6, ca2, cadps, capn6, capsl, ccna1, cdca3, cdh17, celsr2, chrm3, cox11, cpeb1, csf2rb, cx3cr1, cyorf15a, elac2, elavl4, emx2, eps8l3, ern2, esr1, fam167a, fgf9, foxa1, foxg1, gjb6, hlf, hoxa9, hoxc10, hoxd11, hsdl2, htr3a, ibsp, kdelr2, kif13a, kif15, kif2c, klhdc8a, ly6d, ly6e, ly6h, map2k6, meis1, nbla00301, odz1, panx1, pax8, pparg, prame, prdm5, prdm8, prkcq, prkra, pycr1, rax, rgs17, rnls, rtdr1, s100pbp, sdc1, selenbp1, sh2d1a, slc35f2, slc35f5, slc43a1, s1c45a3, slc6a1, slc7a5, sp2, spred2, tmprss3, tmprss4, traj17, trim15, tssc4, upk1b, vgll1, vps33b, wwc1, znf365, and required reagents for making a kit for classification, or in a process for classifying tumor samples.

Attention should be drawn to the fact that although preferred embodiments of the present invention have been described above, it is to be understood that eventual omissions, substitutions and constructive alterations can be carried out by a person skilled in the art without diverting from the spirit and scope of the claimed invention. Further, all combinations of features exerting the same function substantial in the same way to obtain the same results are contemplated by the present invention. Substitutions of features of an embodiment by others are also predicted and contemplated herein.

1. Process for classifying tumor samples of unknown and/or uncertain origin, characterized in that it comprises the steps of:
a) obtaining, from samples of tumors of known origin, the biological activity modulation level of a predetermined group of biomarkers comprising: arf5, batf, c6, ca2, cadps, capn6, ccna1, cdca3, cdh16, cdh17, celsr2, chrm3, cox11, cpeb1, csf2rb, cx3cr1, elac2, elavl4, emx2, eps8l3, ern2, esr1, fgf9, foxa1, foxa1, hlf, hoxa9, hoxc10, hoxd11, hsdl2, htr3a, ibsp, kncj12, kdelr2, kif13a, kif15, kif2c, klhdc8a, ly6d, ly6e, ly6h, map2k6, meis1, nbla00301, odz1, panx1, pax8, pparg, prame, prdm5, prdm8, prkcq, prkra, pycr1, rax, rgs17, rtdr1, s100pbp, sdc1, selenbp1, slc35f2, slc35f5, slc43a1, slc6a1, slc7a5, sp2, spred2, stc1, tmprss3, tmprss4, traj17, trim15, tshr, tssc4, upk1b, vgll1, vps33b, wwc1, znf365, nkx2-1, bcl11b, sh2d1a, prm1, elfn2, slc45a3, fam167a, gjb6, mls, lamp2, capsl, cyorf15a, c14orf105, gfap, fga and stc2;
b) determining from tumor samples of unknown and/or uncertain origin, the biological activity modulation level of the same predetermined group of biomarkers used in step a);
c) normalizing the biological activity modulation level of biomarkers of a) and b) to obtain the ratio between each discriminating biomarker and each normalizing biomarker.
d) comparing the profiles of the biological activity modulation level of the biomarkers of tumor samples of known origin to the profiles of biological activity level of biomarkers of tumor samples of unknown and/or uncertain origin to classify the sample.

2. Process, in accordance with claim 1, characterized in that the samples of tumors of known origin are virtual, wherein virtual samples refers to the data concerning the information of the biological activity of genes of interest which is obtained from pre-established databases.

3. Process, in accordance with claim 1, characterized in that the samples of unknown and/or uncertain origin are real.

4. Process, in accordance with claim 1, characterized in that in that the samples of tumors of known origin are obtained from analysis or experiments of DNA microarrays and/or Real-Time PCR.

5. Process, in accordance with claim 1, characterized in that breast, uterus and/or ovary cancer tumor types are excluded when obtaining profiles of biological activity modulation level of biomarkers which will be compared to unknown and/or uncertain tumor samples obtained from male patients.

6. Process, in accordance with claim 1, characterized in that prostate cancer tumor type is excluded when obtaining profiles of biological activity modulation level of biomarkers which will be compared to unknown and/or uncertain tumor samples of female patients.

7. Process, in accordance with claim 1, characterized in that it comprises using in step c) normalizing biomarkers for carrying out normalization of the biological activity modulation of tumors of known origin and tumors of unknown and/or uncertain origin.

8. Process, in accordance with claim 7, characterized in that it uses 4 normalizing biomarkers in step c), wherein (1) is arf5, (2) is sp2, (3) is vps33b and additionally (4) one biomarker selected from the group consisting of: kdelr2 or ly6e or panx1.

9. Process, in accordance with claim 1, characterized in that the comparison between the data of tumor samples of known origin and the data of tumor samples of unknown and/or uncertain origin is performed by using computational tools.

10. Process, in accordance with claim 9, characterized in that “Random Forest” algorithm is used to relate the data of samples of known origin to the samples of primary or metastatic tumors in order to classify the tumor samples of unknown and/or uncertain origin.

11. Process, in accordance with claim 1, characterized in that said tumor samples are additionally subjected to a quality control process of tumor biological samples to select high quality samples which will be used for generating profiles of their biological activity.

12. Apparatus or system for classification of tumor samples of unknown and/or uncertain origin, characterized in that it comprises means for performing said process for classifying primary or metastatic tumor samples of unknown and/or uncertain origin as defined in claim 1.

13. Quality control process of tumor biological samples of known origin to obtain profiles of biological activity modulation level of biomarkers of tumor samples of known origin in a process for classifying tumor samples, characterized in that it comprises the steps of:
A. subjecting the samples obtained from a pre-selection by the following evaluation criteria:
i. determine if the sample is of origin different from laboratorial or xenotransplant cell lines;
ii. determine if the sample is free of any cancer-related treatment;
iii. determine if the sample is a tumor sample;
iv. determine if the primary origin of the tumor sample is known;
v. determine if the sample is a human (Homo sapiens) sample;
wherein the sample that had all evaluation criteria questions answered positively is pre-selected to be used as a virtual biological sample of high quality, wherein virtual samples refers to the data concerning the information of the biological activity of genes of interest which is obtained from pre-established databases;
B. selecting once more among the samples selected in A. those samples comprising the following group of biomarkers: arf5, batf, c6, ca2, cadps, capn6, ccna1, cdca3, cdh16, cdh17, celsr2, chrm3, cox11, cpeb1, csf2rb, cx3cr1, elac2, elavl4, emx2, eps8l3, ern2, ear1, fgf9, foxa1, foxg1, hlf, hoxa9, hoxc10, hoxd11, hsdl2, htr3a, ibsp, kncj12, kdelr2, kif13a, kif15, kif2c, klhdc8a, ly6d, ly6e, ly6h, map2k6, meis1, nbla00301, odz1, panxl, pax8, pparg, prame, prdm5, prdm8, prkcq, prkra, pycr1, rax, rgs17, rtdr1, s100pbp, sdc1, selenbp1, slc35f2, slc35f5, slc43a1, slc6a1, slc7a5, sp2, spred2, stc1, tmprss3, tmprss4, traj17, trim15, tshr, tssc4, upk1b, vgll1, vps33b, wwc1, znf365, nkx2-1, bcl11b, sh2d1a, prm1, elfn2, slc45a3, fam167a, gjb6, mls, lamp2, capsl, cyorf15a, c14orf105, gfap, fga and stc2;
C. selecting from the group of biomarkers described in B. at least three genes having low variation coefficient among all the analyzed tumor samples;
D. using said at least three biomarkers selected from C) as quality control parameter, satisfying the following relation therebetween:
0.01<[(Biomarker+Biomarker)/2]/Biomarker<10.00;
wherein in case the sample data fall within the range mentioned above, said sample is selected as being a high quality tumor sample of known origin.

14. Quality control process, in accordance with claim 13, characterized in that the group of biomarkers comprise the following relation:
0.01<[(Biomarker_1+Biomarker_2)/2]/Biomarker_3<8.2; and/or
0.07<[(Biomarker_1+Biomarker_3)/2]/Biomarker_2<1.5; and/or
0.61<[(Biomarker_2+Biomarker_3)/2]/Biomarker_1<8.85.;

15. Quality control process, in accordance with claim 13, characterized in that the biomarkers are: /y6e, kdelr2, and panx1.

16. Quality control process, in accordance with claim 15, characterized in that it is used for selecting samples for the process of classifying tumor samples of unknown and/or uncertain origin, and further characterized in that it comprises the steps of:
a) obtaining, from samples of tumors of known origin, the biological activity modulation level of a predetermined group of biomarkers comprising: arf5, batf, c6, ca2, cadps, capn6, ccna1, cdca3, cdh16, cdh17, celsr2, chrm3, cox11, cpeb1, csf2rb, cx3cr1, elac2, elavl4, emx2, eps8l3, ern2, esr1, fgf9, foxa1, foxa1, hlf, hoxa9, hoxc10, hoxd11, hsdl2, htr3a, ibsp, kncj12, kdelr2, kif13a, kif15, kif2c, klhdc8a, ly6d, ly6e, ly6h, map2k6, meis1, nbla00301, odz1, panx1, pax8, pparg, prame, prdm5, prdm8, prkcq, prkra, pycr1, rax, rgs17, rtdr1, s100pbp, sdc1, selenbp1, slc35f2, slc35f5, slc43a1, slc6a1, slc7a5, sp2, spred2, stc1, tmprss3, tmprss4, traj17, trim15, tshr, tssc4, upk1b, vgll1, vps33b, wwc1, znf365, nkx2-1, bcl11b, sh2d1a, prm1, elfn2, slc45a3, fam167a, gjb6, mls, lamp2, capsl, cyorf15a, c14orf105, gfap, fga and stc2;
b) determining from tumor samples of unknown and/or uncertain origin, the biological activity modulation level of the same predetermined group of biomarkers used in step a):
c) normalizing the biological activity modulation level of biomarkers of a) and b) to obtain the ratio between each discriminating biomarker and each normalizing biomarker.
d) comparing the profiles of the biological activity modulation level of the biomarkers of tumor samples of known origin to the profiles of biological activity level of biomarkers of tumor samples of unknown and/or uncertain origin to classify the sample.

17. Quality control process of biological samples of unknown and/or uncertain origin to obtain profiles of biological activity modulation level of biomarkers of tumor samples of unknown and/or uncertain origin in a process for classifying tumor samples, characterized in that it comprises the steps of:
I) processing the samples obtained for extraction and purification of analytes of the biological material;
II) subjecting the analytes to amplification in which collection of data of the respective amplification cycles (Ct) is carried out;
III) the sample of II) must be submitted to the following evaluation criterion:
Ct 10.00<Ct value of the analyzed biomarker <Ct 40.00;
wherein in case the sample falls within the range mentioned above, the sample is selected as being a real sample of high quality.

18. Control process, in accordance with claim 17, characterized in that the samples are subjected to the following evaluation criteria:
1) Ct 18.00<ARF5<Ct 25.52;
2) Ct 15.63<SP2<Ct 31.63;
3) Ct 16.48<KDELR2<Ct25.53;
4) Ct 19.58<LYE6<Ct29.34;
5) Ct 18.16<PANX1<Ct27.46;
and additionally the samples selected in accordance the criteria 1 to 5 being subjected to the following evaluation criteria:
6) Ct24.37<VPS33B<Ct 35.76—only if outside the range, replace by Ct27.52;
7) Ct 25.53<TSSC4<Ct34.90—only if outside the range, replace by Ct29.40.

19. Quality control process, in accordance with claim 17, characterized in that the used biomarker(s) is one or more biomarkers selected from the group comprising:
arf5, sp2, vpss33b, tssc4, kdelr2, lye6 and panx1.

20. Quality control process, in accordance with claim 17, characterized in that it is used for selecting samples for the process for classifying tumor samples of unknown and/or uncertain origin; and further characterized in that it comprises the steps of:
a) obtaining, from samples of tumors of known origin, the biological activity modulation level of a predetermined group of biomarkers comprising: arf5, batf, c6, ca2, cadps, capn6, ccna1, cdca3, cdh16, cdh17, celsr2, chrm3, cox11, cpeb1, csf2rb, cx3cr1, elac2, elavl4, emx2, eps8l3, ern2, esr1, fgf9, foxa1, foxa1, hlf, hoxa9, hoxc10, hoxd11, hsdl2, htr3a, ibsp, kncj12, kdelr2, kif13a, kif15, kif2c, klhdc8a, ly6d, ly6e, ly6h, map2k6, meis1, nbla00301, odz1, panx1, pax8, pparg, prame, prdm5, prdm8, prkcq, prkra, pycr1, rax, rgs17, rtdr1, s100pbp, sdc1, selenbp1, slc35f2, slc35f5, slc43a1, slc6a1, slc7a5, sp2, spred2, stc1, tmprss3, tmprss4, traj17, trim15, tshr, tssc4, upk1b, vgll1, vps33b, wwc1, znf365, nkx2-1, bcl11b, sh2d1a, prm1, elfn2, slc45a3, fam167a, gjb6, mls, lamp2, capsl, cyorf15a, c14orf105, gfap, fga and stc2;
b) determining from tumor samples of unknown and/or uncertain origin, the biological activity modulation level of the same predetermined group of biomarkers used in step a);
c) normalizing the biological activity modulation level of biomarkers of a) and b) to obtain the ratio between each discriminating biomarker and each normalizing biomarker.
d) comparing the profiles of the biological activity modulation level of the biomarkers of tumor samples of known origin to the profiles of biological activity level of biomarkers of tumor samples of unknown and/or uncertain origin to classify the sample.

21. Kit for classification of tumor samples of unknown and/or uncertain origin by using the process as defined in claim 1, characterized in that it comprises means for identifying and classifying tumor samples, comprising reagents for identifying the biological activity level of the following biomarkers: arf5, batf, c6, ca2, cadps, capn6, ccna1, cdca3, cdh16, cdh17, celsr2, chrm3, cox11, cpeb1, csf2rb, cx3cr1, elac2, elavl4, emx2, eps8l3, ern2, esr1, fgf9, foxa1, foxa1, hlf, hoxa9, hoxc10, hoxd11, hsdl2, htr3a, ibsp, kncj12, kdelr2, kif13a, kif15, kif2c, klhdc8a, ly6d, ly6e, ly6h, map2k6, meis1, nbla00301, odz1, panx1, pax8, pparg, prame, prdm5, prdm8, prkcq, prkra, pycr1, rax, rgs17, rtdr1, s100pbp, sdc1, selenbp1, slc35f2, slc35f5, slc43a1, slc6a1, slc7a5, sp2, spred2, stc1, tmprss3, tmprss4, traj17, trim15, tshr, tssc4, upk1b, vgll1, vps33b, wwc1, znf365, nkx2-1, bcl11b, sh2d1a, prm1, elfn2, slc45a3, fam167a, gjb6, mls, lamp2, capsl, cyorf15a, c14orf105, gfap, fga and stc2;

22. Kit, in accordance with claim 21, characterized in that it further comprises at least one reagent that specifically binds to the biomarkers and/or at least an electronic device for processing information about biological activity of said biomarkers.

23. Use of genes as a group of biomarkers, characterized by the genes are used in the manufacture of a kit for classification or in a process for classifying tumor samples, wherein such genes consist of cdh16, fga, gfap, kcnj12, nkx2-1, prm1, tshr, elfn2, lamp2, stc1, stc2 and at least one of arf5, batf, bcl11b, c14orf105, c6, ca2, cadps, capn6, capsl, ccna1, cdca3, cdh17, celsr2, chrm3, cox11, cpeb1, csf2rb, cx3cr1, cyorf15a, elac2, elavl4, emx2, eps8l3, ern2, esr1, fam167a, fgf9, foxa1, foxg1, gjb6, hlf, hoxa9, hoxc10, hoxd11, hsdl2, htr3a, ibsp, kdelr2, kif13a, kif15, kif2c, klhdc8a, ly6d, ly6e, ly6h, map2k6, meis1, nbla00301, odz1, panx1, pax8, pparg, prame, prdm5, prdm8, prkcq, prkra, pycr1, rax, rgs17, mls, rtdr1, s100pbp, sdc1, selenbp1, sh2d1a, s1c35f2, s1c35f5, slc43al, s1c45a3, slc6al, slc7a5, sp2, spred2, tmprss3, tmprss4, traj17, trim15, tssc4, upk1b, vgll1, vps33b, wwc1, znf365.
